(19) United States
(12) Patent Application Publication
(10) Pub. No.: US 2024/0415835 A1
(43) Pub. Date: Dec. 19, 2024

(54) COMPOSITIONS COMPRISING AN ERK INHIBITOR

(71) Applicant: Otsuka Pharmaceutical Co., Ltd., Tokyo (JP)

(72) Inventors: Aaron Goodwin, Bend, OR (US); Sindhuri Maddineni, Dublin, CA (US); Nipun Davar, Pleasanton, CA (US)

(21) Appl. No.: 18/702,752

(22) PCT Filed: Oct. 25, 2022
(86) PCT No.: PCT/US2022/047769

§ 371 Date: Apr. 18, 2024

Related U.S. Application Data
(60) Provisional application No. 63/271,977, filed on Oct. 26, 2021.

(51) Int. Cl. A61K 31/506; A61K 9/10; A61K 9/20; A61K 9/28; A61K 47/38

(52) U.S. Cl. CPC A61K 31/506; A61K 9/10; A61K 9/2009; A61K 9/2013; A61K 9/2018; A61K 9/2054; A61K 9/28; A61K 47/38

(57) ABSTRACT

The present disclosure relates generally to compositions comprising Compound (I).

### CROSS REFERENCE TO RELATED APPLICATIONS

This application claims the benefit under 35 U.S.C. § 119 (e) of U.S. Provisional Application No. 63/271,977 filed Oct. 26, 2021, which is hereby incorporated by reference herein in its entirety.

### FIELD

The present application relates to compositions of an ERK1/2 inhibitor.

### BACKGROUND

The extracellular signal regulated kinases (ERK1/2) are ubiquitously expressed protein serine/threonine kinases that comprise a key component of the mitogen-activated protein kinase (MAPK) signaling pathway. The MAPK pathway is an evolutionary conserved cell signaling pathway that regulates a variety of cellular processes including cell cycle progression, cell migration, cell survival, differentiation, metabolism, proliferation and transcription. ERK1/2 activity is commonly upregulated in cancer, as a result of activating mutations within upstream components of the MAPK pathway. ERK1/2 inhibitors are useful in therapy, in particular in the treatment of cancer.

There is a need for compositions comprising ERK1/2 inhibitor compounds which are stable and/or easy to formulate and/or have improved bioavailability and/or improved pharmacokinetic profiles. The bioavailability of active ingredients can impact therapeutic efficacy.

### SUMMARY

The present disclosure, in one embodiment, provides compositions comprising Compound I, or a pharmaceutically acceptable salt or solvate thereof:

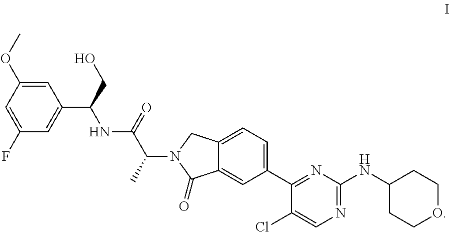 [SMILES: COc1cc(F)cc([C@@H](CO)NC(=O)[C@@H](C)N2Cc3ccc(-c4nc(NC5CCOCC5)ncc4Cl)cc3C2=O)c1]

Compound I is named (2R)-2-(6-{5-chloro-2-[(tetrahydro-2H-pyran-4-yl)amino]pyrimidin-4-yl}-1-oxo-2,3-dihydro-1H-isoindol-2-yl)-N-[(S)-1-(3-fluoro-5-methoxyphenyl)-2-hydroxyethyl]propanamide. Provided herein, in one aspect, is a pharmaceutical composition comprising Compound I, having the formula:

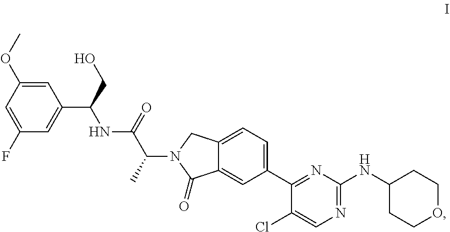 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof, and a water soluble polymer.

In one aspect, provided herein is a solid dispersion comprising Compound I, having the formula:

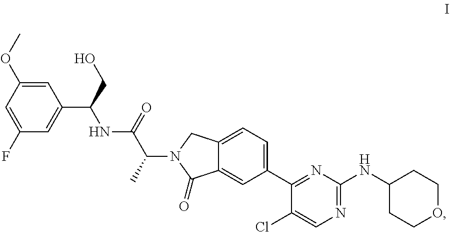 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS).

In another aspect, provided herein is a tablet comprising a solid dispersion, wherein the solid dispersion comprises Compound I, having the formula:

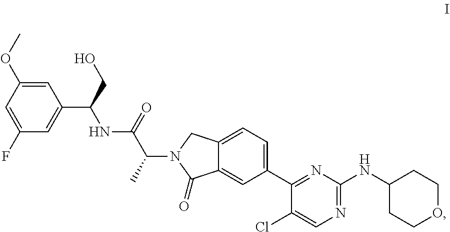 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS); and the ratio of an amount by weight of Compound I, or a pharmaceutically acceptable salt or solvate thereof, in the solid dispersion to an amount by weight of HPMCAS in the solid dispersion is about 1:3.

Also provided herein is the use of any composition comprising Compound I described herein, or a pharmaceutically acceptable salt or solvate thereof, for treating cancer. Further provided is a method of treating cancer comprising administering a composition comprising Compound I, or a pharmaceutically acceptable salt or solvate thereof, described herein to an individual in need thereof.

### BRIEF DESCRIPTION OF THE DRAWINGS

Embodiments of the present application are described, by way of example only, with reference to the attached Figures, wherein:

FIG. 1 shows dissolution profiles for certain compositions of Compound I.

FIG. 2 shows drug speciation data after dissolution tests for certain compositions of Compound I.

FIG. 3 shows dissolution profiles for certain compositions of Compound I.

FIG. 4A shows plasma concentrations after administration of Compound I free base hydrate formulations in a dog pharmacokinetic (PK) study as described in Example 4. The error bars refer to standard deviation.

FIG. 4B shows a comparison of area under the curve (AUC) after administration of Compound I free base hydrate formulations in a dog PK study as described in Example 4. The error bars refer to standard deviation.

FIG. 4C shows AUC in a dog pharmacokinetic (PK) study as described in Example 4.

FIG. 5A shows AUCₗₐₛₜ after administration of Compound I free base hydrate formulations in a primate PK study.

FIG. 5B shows Cₘₐₓ after administration of Compound I free base hydrate formulations in a primate PK study.

FIG. 5C shows a comparison of area under the curve (AUC) after administration of Compound I free base hydrate formulations in a primate PK study.

### DETAILED DESCRIPTION

Compound I has been described as Example 685 in WO 2017/068412, which reference is incorporated herein by reference in its entirety. Compound I is useful for the treatment of cancer and other conditions described in WO 2017/068412. In formulating Compound I, the relative amounts based on weight of Compound I and weight of a polymer within a solid dispersion were found to have an unexpected impact on the bioavailability of Compound I.

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O) NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. −(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl). 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

As used herein “hydroxypropylmethyl cellulose acetate succinate” may also be referred to interchangeably as HPMCAS or hypromellose acetate succinate (e.g., Aquasolve™, Shin-Etsu AQOAT®, or other commercial equivalents). HPMCAS may have various molecular weights (e.g., 10,000 to 500,000 da), and is commercially available in different grades (e.g., L, M, or H) that vary in the extent of substitutions of acetyl and succinoyl groups and particle size (e.g., For G). For instance, the L grade of Aquasolve™ is commercially sold and comprises 5-9% acetyl content, 14-18% succinoyl content, 20-24% methoxyl content, and 5-9% hydroxypropoxy content. For instance, the M grade of Aquasolve™ is commercially sold and comprises 7-11% acetyl content, 10-14% succinoyl content, 21-25% methoxyl content, and 5-9% hydroxypropoxy content. For instance, the S grade of Aquasolve™ is commercially sold and comprises 10-14% acetyl content, 4-8% succinoyl content, 22-26% methoxyl content, and 6-10% hydroxypropoxy content. Each grade is available in fine (F) and granular (G) particle sizes. In some instances, fine (F) types will have average particle size of 10 microns or lower, while granular (G) types will have average particle size of 20 microns or lower.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds may exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of the compound of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal, particularly a human. See, for example, Foster, “Deuterium Isotope Effects in Studies of Drug Metabolism.” Trends Pharmacol. Sci. 5 (12): 524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto. A “salt” may be derived from an inorganic acid, an inorganic base, an organic acid, or an organic base. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, mandelic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, tetrahydrofuran carboxylic acid, and the like. Salts derived from inorganic bases include, by way of example only, sodium, potassium, lithium, ammonium, calcium and magnesium salts. Salts derived from organic bases include, but are not limited to, salts of primary, secondary, and tertiary amines.

Provided are also compositions comprising pharmaceutically acceptable salts, hydrates, solvates, and tautomeric forms of Compound I described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl)amines (i.e., HN(substituted alkyl)₂), tri(substituted alkyl)amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

A free base, a salt, or a pharmaceutically acceptable salt provided herein may be a “solvate” formed by the interaction of a solvent and a compound. Solvates of salts and the free base of Compound I described herein are provided. Where the solvent is water, the solvate is a hydrate. A salt or pharmaceutically acceptable salt provided herein may be a hydrate. “Hydrates” of Compound I free base described herein are also provided. In some embodiments the hydrate is a monohydrate.

### Compositions

Provided herein is a pharmaceutical composition comprising Compound I having the formula:

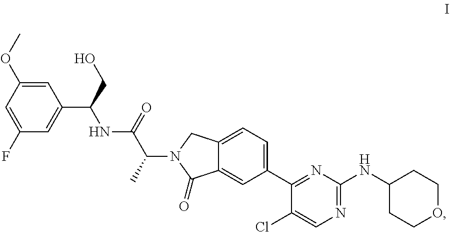 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof, and a water soluble polymer.

In some embodiments, the water soluble polymer is polyvinylpyrrolidone/vinyl acetate (PVPVA), hydroxypropylmethyl cellulose (HPMC), or any grade of hydroxypropylmethyl cellulose acetate succinate (HPMCAS). In some embodiments, the water soluble polymer is HPMCAS. In some embodiments, the grade of HPMCAS is L and comprises 5-9% acetyl content, 14-18% succinoyl content, 20-24% methoxyl content, and 5-9% hydroxypropoxy content. In some embodiments, the grade of HPMCAS is M and comprises 7-11% acetyl content, 10-14% succinoyl content, 21-25% methoxyl content, and 5-9% hydroxypropoxy content. In some embodiments, the grade of HPMCAS is S and comprises 10-14% acetyl content, 4-8% succinoyl content, 22-26% methoxyl content, and 6-10% hydroxypropoxy content. In any of these embodiments, the HPMCAS has a fine particle size such as the F particle size available commercially for Aquasolve™. In any of these embodiments, the HPMCAS has a granular particle size such as the G particle size available commercially for Aquasolve™.

In some embodiments, the composition comprises Compound I free base hydrate. In some or any embodiments provided herein, the composition comprises Compound I free base monohydrate. In some or any embodiments provided herein, the composition comprises Compound I Form B as described in WO 2018/193410.

Further provided herein is a solid dispersion comprising Compound I, having the formula:

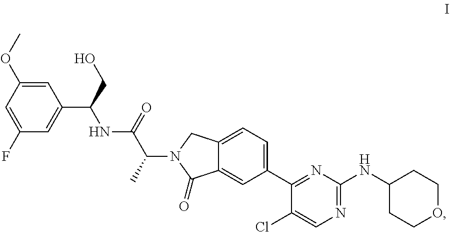 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS).

In some embodiments, Compound I, or a pharmaceutically acceptable salt or solvate thereof, is present in the solid dispersion in an amount of about 10% to about 50% by weight of the solid dispersion. In other words, in some embodiments, for every 100 g of the solid dispersion, 10-50 g of the total weight of the solid dispersion is from the weight of Compound I, or a pharmaceutically acceptable salt or solvate thereof, and 90-50 g of the total weight of the solid dispersion is from the weight of HPMCAS. In some embodiments, Compound I, or a pharmaceutically acceptable salt or solvate thereof, is present in the solid dispersion in an amount of about 20% to about 50% by weight of the solid dispersion. In some embodiments, Compound I, or a pharmaceutically acceptable salt or solvate thereof, is present in the solid dispersion in an amount of about 20% to about 30% by weight of the solid dispersion.

In some embodiments, Compound I, or a pharmaceutically acceptable salt or solvate thereof, is substantially amorphous. In some embodiments, within the solid dispersion, at least 98% of Compound I, or a pharmaceutically acceptable salt or solvate thereof, is in amorphous form. In some embodiments, within the solid dispersion, at least 95% of Compound I, or a pharmaceutically acceptable salt or solvate thereof, is in amorphous form. In some embodiments, within the solid dispersion, at least 90% of Compound I, or a pharmaceutically acceptable salt or solvate thereof, is in amorphous form. In some embodiments, within the solid dispersion, Compound I is a free base or a solvate thereof. In some embodiments, within the solid dispersion, Compound I is a free base hydrate.

In some embodiments, the weight ratio of Compound I, or a pharmaceutically acceptable salt or solvate thereof, to HPMCAS in the solid dispersion is from about 1:1 to about 1:5. In some embodiments, the weight ratio of Compound I, or a pharmaceutically acceptable salt or solvate thereof, to HPMCAS is from about 1:2 to about 1:4. In some embodiments, the weight ratio of Compound I, or a pharmaceutically acceptable salt or solvate thereof, to HPMCAS is about 1:3. In some embodiments, the weight ratio of Compound I, or a pharmaceutically acceptable salt or solvate thereof, to HPMCAS is about 1:2. In some embodiments, the weight ratio of Compound I, or a pharmaceutically acceptable salt or solvate thereof, to HPMCAS is about 1:1.

In some embodiments, the HPMCAS is present in an amount greater than about 50% by weight of the solid dispersion. In some embodiments, the HPMCAS is present in an amount greater than about 60% by weight of the solid dispersion. In some embodiments, the HPMCAS is present in an amount greater than about 70% by weight of the solid dispersion.

In some embodiments, the HPMCAS is present in an amount of about 60% to about 80% by weight of the solid dispersion. In some embodiments, the HPMCAS is present in an amount of about 70% to about 80% by weight of the solid dispersion.

In some embodiments, the HPMCAS is present in an amount of about 50% by weight of the solid dispersion. In some embodiments, the HPMCAS is present in an amount of about 60% by weight of the solid dispersion. In some embodiments, the HPMCAS is present in an amount of about 70% by weight of the solid dispersion. In some embodiments, the HPMCAS is present in an amount of about 75% by weight of the solid dispersion.

In some embodiments, the HPMCAS is present in an amount of about 50% by weight of the solid dispersion and Compound I, or a pharmaceutically acceptable salt or solvate thereof, is present in an amount of about 50% by weight of the solid dispersion. In some embodiments, the HPMCAS is present in an amount of about 60% by weight of the solid dispersion and Compound I, or a pharmaceutically acceptable salt or solvate thereof, is present in an amount of about 40% by weight of the solid dispersion. In some embodiments, the HPMCAS is present in an amount of about 70% by weight of the solid dispersion and Compound I, or a pharmaceutically acceptable salt or solvate thereof, is present in an amount of about 30% by weight of the solid dispersion. In some embodiments, the HPMCAS is present in an amount of about 75% by weight of the solid dispersion and Compound I, or a pharmaceutically acceptable salt or solvate thereof, is present in an amount of about 25% by weight of the solid dispersion.

In some or any embodiments, the HPMCAS within the solid dispersion has an acetyl content in a range of about 5 percent to about 14 percent by weight, and succinoyl content in a range of about 5 percent to about 20 percent by weight of the HPMCAS. In some embodiments, the HPMCAS within the solid dispersion has an acetyl content in a range of about 7 percent to about 11 percent by weight, and succinoyl content in a range of about 10 to about 14 percent by weight of the HPMCAS.

In some embodiments of the tablet, the solid dispersion comprises about 40-70% by weight of the tablet. In some embodiments of the tablet, the solid dispersion comprises about 40%, about 45%, about 50%, about 55%, about 60%, about 65%, or about 70% by weight of the tablet. In some embodiments of the tablet, the solid dispersion comprises about 60% by weight of the tablet.

Provided herein is a pharmaceutical composition comprising a solid dispersion described herein. In some embodiments, the composition is formulated as a tablet. In some embodiments, the composition is formulated as a tablet comprising an intra-granular layer and an extra-granular layer. In some of such embodiments, the solid dispersion is in the intra-granular layer and the intra-granular layer further comprises additional excipients as described herein. In some of such embodiments, the solid dispersion is in the extra-granular layer and the extra-granular layer further comprises additional excipients as described herein. In some of such embodiments, the solid dispersion is in the intra-granular layer and in the extra-granular layer and the intra-granular layer and/or the extra-granular layer further comprises additional excipients as described herein.

In some embodiments of the pharmaceutical composition formulated as a tablet, the intra-granular layer of the tablet comprises about 8-18%, about 10-18% or about 12-18% by weight of microcrystalline cellulose. In some embodiments of the pharmaceutical composition formulated as a tablet, the intra-granular layer of the tablet further comprises about 8-18%, about 10-18% or about 12-18% by weight of lactose. In some embodiments, the lactose is lactose monohydrate. In some embodiments, the lactose monohydrate is agglomerated lactose monohydrate (e.g., Tablettose®). In some embodiments of the pharmaceutical composition formulated as a tablet, the intra-granular layer of the tablet further comprises about 2-8%, about 4-8% or about 6-8% by weight of croscarmellose sodium. In some embodiments of the pharmaceutical composition formulated as a tablet, the intra-granular layer of the tablet further comprises about 0.5-2%, or about 0.5-1.5% by weight of non-fumed silica. In some embodiments of the pharmaceutical composition formulated as a tablet, the intra-granular layer of the tablet further comprises about 0.1-0.5% or about 0.2-0.4% by weight of magnesium stearate.

In some embodiments of the pharmaceutical composition formulated as a tablet, the intra-granular layer of the tablet comprises:
about 8-18% by weight of microcrystalline cellulose;
about 8-18% by weight of lactose monohydrate;
about 2-8% by weight of croscarmellose sodium;
about 0.5-2% by weight of non-fumed silica; and
about 0.1-0.5% by weight of magnesium stearate.

In some embodiments, the % by weight amounts of microcrystalline cellulose, lactose monohydrate, croscarmellose sodium, non-fumed silica and/or magnesium stearate in the intra-granular layer are as described in any embodiments described above or in the Examples section. In some of these embodiments, the solid dispersion is in the intra-granular layer. In some of these embodiments, the solid dispersion is in the extra-granular layer. In some of these embodiments, the solid dispersion is in the intra-granular layer and in the extra-granular layer.

In some embodiments of the pharmaceutical composition formulated as a tablet, the extra-granular layer of the tablet comprises about 0-14%, or about 5-14% by weight of microcrystalline cellulose. In some embodiments, the solid dispersion is in the intra-granular layer and the extra-granular layer does not comprise any microcrystalline cellulose. In some embodiments of the pharmaceutical composition formulated as a tablet, the extra-granular layer of the tablet comprises about 0-14%, or about 5% to about 10% by weight of lactose. In some embodiments, the lactose is lactose monohydrate. In some embodiments, the lactose is agglomerated lactose monohydrate (e.g., Tablettose®). In some embodiments, the solid dispersion is in the intra-granular layer and the extra-granular layer does not comprise any lactose.

In some embodiments of the pharmaceutical composition formulated as a tablet, the extra-granular layer of the tablet comprises about 2-8%, or about 3-5% by weight of croscarmellose sodium. In some embodiments of the pharmaceutical composition formulated as a tablet, the extra-granular layer of the tablet comprises about 0.5-2%, or about 0.5-1.5% by weight of non-fumed silica. In some embodiments of the pharmaceutical composition formulated as a tablet, the extra-granular layer of the tablet comprises about 0.1-0.5%, or about 0.2-0.4% by weight of magnesium stearate.

In some embodiments of the pharmaceutical composition formulated as a tablet, the extra-granular layer of the tablet comprises:
about 0-14% by weight of microcrystalline cellulose;
about 0-14% by weight of lactose monohydrate;
about 2-8% by weight of croscarmellose sodium;
about 0.5-2% by weight of non-fumed silica; and
about 0.1-0.5% by weight of magnesium stearate.

In some embodiments of the pharmaceutical composition formulated as a tablet, the extra-granular layer of the tablet comprises:
about 2-8% by weight of croscarmellose sodium;
about 0.5-2% by weight of non-fumed silica; and
about 0.1-0.5% by weight of magnesium stearate.

In some embodiments, the % by weight amounts of microcrystalline cellulose, lactose monohydrate, croscarmellose sodium, non-fumed silica and/or magnesium stearate in the extra-granular layer are as described in any embodiments described above or in the Examples section. In some of these embodiments, the solid dispersion is in the intra-granular layer. In some of these embodiments, the solid dispersion is in the extra-granular layer. In some of these embodiments, the solid dispersion is in the intra-granular layer and in the extra-granular layer.

In some embodiments of the pharmaceutical composition formulated as a tablet, the table is coated with a film. In some embodiments, the film coat comprises a polymer, a plasticizer and/or a pigment. Examples of polymers in a tablet film coating include and are not limited to polyvinyl alcohol (PVA), polyvinylpyrrolidone (copovidone), hydroxypropylmethyl cellulose (HPMC), methacrylic acid copolymer, methyl cellulose, ethyl cellulose or any other suitable film forming polymer. Examples of plasticizers in a tablet film coating include and are not limited to hydroxypropylmethyl cellulose (HPMC), polyethylene glycol, triacetin, stearates, citrates, phthalate esters or any other suitable plasticizers. In some embodiments, the film coating is OPADRY II®.

In one aspect, provided herein is a tablet comprising a solid dispersion, wherein the solid dispersion comprises Compound I, having the formula:

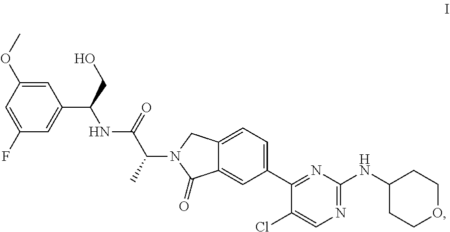 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS); and the ratio of an amount by weight of Compound I, or a pharmaceutically acceptable salt or solvate thereof, in the solid dispersion to an amount by weight of HPMCAS in the solid dispersion is about 1:3. In some other embodiments of the tablet, the ratio of an amount by weight of Compound I, or a pharmaceutically acceptable salt or solvate thereof, in the solid dispersion to an amount by weight of HPMCAS in the solid dispersion is about 1:2, or about 1:1.

In some embodiments of the tablet, Compound I, or a pharmaceutically acceptable salt or solvate thereof, is substantially amorphous. In some embodiments of the tablet, at least 98% of Compound I, or a pharmaceutically acceptable salt or solvate thereof, is in amorphous form. In some embodiments of the tablet, at least 95% of Compound I, or a pharmaceutically acceptable salt or solvate thereof, is in amorphous form. In some embodiments of the tablet, at least 90% of Compound I, or a pharmaceutically acceptable salt or solvate thereof, is in amorphous form. In some embodiments of the tablet, Compound I is a free base or a solvate thereof. In some embodiments of the tablet, Compound I is a free base hydrate.

In some embodiments, provided herein is a tablet composition of Compound I having the formula:

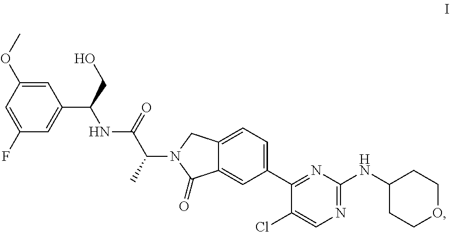 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
comprising:
an intra-granular portion comprising:
   about 40-80% by weight of a solid dispersion of Compound I, or a pharmaceutically acceptable salt or solvate thereof, and HPMCAS, wherein Compound I, or a pharmaceutically acceptable salt or solvate thereof, is substantially amorphous and molecularly dispersed within a polymer matrix comprising hydroxypropylmethyl cellulose acetate succinate (HPMCAS), and the ratio of an amount by weight of Compound I, or a pharmaceutically acceptable salt or solvate thereof, in the solid dispersion to an amount by weight of HPMCAS in the solid dispersion is about 1:3;
   about 8-18% by weight of microcrystalline cellulose;
   about 8-18% by weight of lactose monohydrate;
   about 2-8% by weight of croscarmellose sodium;
   about 0.5-2% by weight of non-fumed silica; and
   about 0.1-0.5% by weight of magnesium stearate,
and
an extra-granular portion comprising:
   about 2-8% by weight of croscarmellose sodium;
   about 0.5-2% by weight of non-fumed silica; and
   about 0.1-0.5% by weight of magnesium stearate.

In another aspect, provided herein is a tablet composition of Compound I having the formula:

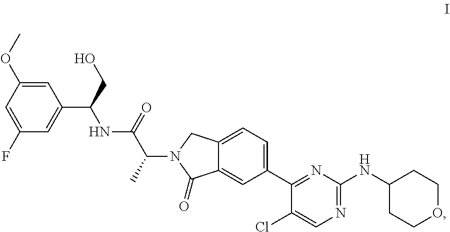 [SMILES: COc1cc(F)cc([C@@H](CO)NC(=O)[C@@H](C)N2Cc3ccc(-c4nc(NC5CCOCC5)ncc4Cl)cc3C2=O)c1]

or a pharmaceutically acceptable salt or solvate thereof,
comprising:
an intra-granular portion comprising:
   about 60% by weight of a solid dispersion of Compound I, or a pharmaceutically acceptable salt or solvate thereof, and HPMCAS, wherein Compound I, or a pharmaceutically acceptable salt or solvate thereof, is substantially amorphous and molecularly dispersed within a polymer matrix comprising hydroxypropylmethyl cellulose acetate succinate (HPMCAS), and the ratio of an amount by weight of Compound I, or a pharmaceutically acceptable salt or solvate thereof, in the solid dispersion to an amount by weight of HPMCAS in the solid dispersion is about 1:3;
   about 14% by weight of microcrystalline cellulose;
   about 13.5% by weight of lactose monohydrate;
   about 6% by weight of croscarmellose sodium;
   about 1% by weight of non-fumed silica; and
   about 0.25% by weight of magnesium stearate;
and
an extra-granular portion comprising:
   about 4% by weight of croscarmellose sodium;
   about 1% by weight of non-fumed silica; and
   about 0.25% by weight of magnesium stearate.

In some embodiments of the tablet composition of Compound I, or a pharmaceutically acceptable salt or solvate thereof, the HPMCAS has an acetyl content in a range of about 7% to about 11% by weight, and succinoyl content in a range of about 10% to about 14% by weight of the HPMCAS.

Provided herein is the use of a composition described herein for treating cancer.

Also provided is a method of treating cancer comprising administering a composition of Compound I, or a pharmaceutically acceptable salt or solvate thereof, described herein to an individual in need thereof.

Provided herein is a process for preparing a solid dispersion of Compound I, or a pharmaceutically acceptable salt or solvate thereof, comprising spray drying a solution of Compound I, or a pharmaceutically acceptable salt or solvate thereof, and HPMCAS in a solvent, wherein the solution has a solids loading of up to about 20% by weight. In some embodiments, the solution in has a solids loading of about 5% to about 15%, or about 10%. In some embodiments, the solvent is methanol. In some embodiments, the solvent is acetone. In some embodiments, the solvent is a mixture of methanol and acetone. In some embodiments of the process, Compound I is a free base. In some embodiments of the process, Compound I is a free base monohydrate. In some embodiments, Compound I is Form B as described in WO 2018/193410, where Form B is compound I free base monohydrate.

Provided herein is a process for preparing a solid dispersion of Compound I, or a pharmaceutically acceptable salt or solvate thereof, comprising spray drying a solution of Compound I, or a pharmaceutically acceptable salt or solvate thereof, and HPMCAS in acetone, wherein the solution in acetone has a solids loading of up to about 10% by weight. In some embodiments, the solution in acetone has a solids loading of about 5% to about 10%. In some embodiments of the process, Compound I is a free base. In some embodiments of the process, Compound I is a free base hydrate.

Compound I, or a pharmaceutically acceptable salt or solvate thereof, of this disclosure can be prepared from readily available starting materials using general methods and procedures described in, for example, WO 2017/068412. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure. If available, reagents may be purchased commercially, e.g., from Sigma Aldrich or other chemical suppliers.

### Example 1-1: Preparation of Solid Dispersion of Compound I: HPMCAS 1:3

The amorphous solid dispersion comprises Compound I: HPMCAS (Shin Etsu, M grade, G particle size) in a ratio of 1:3. The solid dispersion was manufactured by spray drying a solution of 2 grams of the free base hydrate crystalline form B of Compound I and 6 grams of HPMCAS, out of acetone with a solids loading of 10% w/w, or optionally a loading of about 4% w/w to about 10% w/w. The free base monohydrate crystalline form B of Compound I was prepared as described in WO 2018/193410. Solid dispersions were prepared using a spray draying process. The spray drying process involved three steps: preparation of feed solution, spray drying and secondary drying. To prepare the feed solution, 90% of the total acetone amount was added to the reactor and then Compound I was added to the reactor. The mixture was stirred at room temperature until the solids dissolved. HPMCAS (Shin Etsu, M grade, G particle size) was added and the mixture was stirred at room temperature until most of the solids dissolved. The remaining amount of acetone was added to the reactor and stirring continued for an additional time (30 minutes or longer) until solids dissolved. For spray drying, the spray dryer (BLD-35 manufactured by Bend) was stabilized with nitrogen gas followed by introduction of the solution, the wet spray was collected in bags, and the collected wet spray dried material was then secondary dried in a vacuum tray till residual solvent is below 1,500 ppm. The spray dried material was an amorphous white to off-white solid, having a yield of 86.8%.

### Example 1-2: Preparation of Solid Dispersion of Compound I: HPMCAS 1:1

The amorphous solid dispersion comprises Compound I: HPMCAS (Shin Etsu, M grade, G particle size) in a ratio of 1:1. The solid dispersion was manufactured by spray drying, from 4 grams of the free base hydrate crystalline form B of Compound I and 4 grams of HPMCAS, using the similar procedure of Example 1-1.

### Example 1-3: Preparation of Solid Dispersion of Compound I: HPMC 1:3

The amorphous solid dispersion comprises Compound I: HPMC (JRS PHARMA, E3 grade) in a ratio of 1:3. The solid dispersion was manufactured by spray drying, from 2 grams of the free base hydrate crystalline form B of Compound I and 6 grams of HPMC, using the similar procedure of Example 1-1, except that 90% methanol (9:1 methanol: water) was used as a solvent for spray drying solution.

### Example 2: Preparation of Tablet Comprising Solid Dispersion

The tablet dosage form was developed using a dry granulation process with 60% w/w amorphous solid dispersion and other excipients. The amorphous solid dispersions, microcrystalline cellulose, lactose monohydrate, silicon dioxide, and croscarmellose sodium were each de-lumped and blended. Magnesium stearate was de-lumped by sieving and blended in. This constituted the intragranular blend. The intragranular blend was roller compacted and milled into granules. The granules, microcrystalline cellulose, lactose monohydrate, silicon dioxide, and croscarmellose sodium were each de-lumped and blended. Magnesium stearate was de-lumped by sieving and blended with the blend from the previous unit operation. This constituted the tablet blend which was prepared on a 12 kg scale using the weight proportions given in Table 2 below. The tablet blend was compressed into tablet cores of desired dosage of the drug on a rotary tablet press. The tablet weight was varied to obtain different dosages of the drug. Different size and shape tooling was used for each dosage strength. The tablet cores were cosmetically coated in a pan coater.

Table 1 below shows the composition of a tablet composition of Compound I comprising the solid dispersion of Example 1.

TABLE 1

| | % w/w |
|---|---|
| Intra-granular | |
| solid dispersion | 60% |
| Microcrystalline cellulose (MCC Ph102) | 14% |
| Lactose monohydrate (Tablettose 80 ®) | 13.5% |
| Croscarmellose sodium (Ac-Di-Sol ®) | 6% |
| Non-fumed Silica | 1% |
| Mg Stearate | 0.25% |
| Extra-granular | |
| Croscarmellose sodium (Ac-Di-Sol ®) | 4% |
| Non-fumed Silica | 1.0% |
| Mg Stearate | 0.25% |

### Example 3-1: Dissolution Study

The solid dispersions described in Example 1 were evaluated in two non-sink dissolution tests to evaluate various aspects of the formulations and how they might be differentiated. The two dissolution tests that were evaluated include a gastric to intestinal (G-IB) transfer microcentrifuge dissolution test and an ultracentrifuge free-drug test.

The gastric-to-intestinal buffer (G-IB) transfer dissolution test is used to measure the supersaturation of drug above the solubility of bulk drug when dosed as a powder, first into media at a lower pH value to imitate a fasted stomach. After 30 minutes of exposure to the gastric media, the samples are transferred into the higher pH media as an imitation of the intestine. The drug concentrations measured in this test are a composite of free drug, drug in micelles, and drug suspended in solution as drug/polymer colloids.

The ultracentrifuge free-drug analysis is used to determine the species of dissolved drug that are present for each formulation. This test is performed at several timepoints, i.e., 0, 30, 60 and 90 minutes, during the microcentrifuge test, and it consists of a centrifugation step at 300,000 g that removes any colloidal species that are present, leaving only the free drug and drug in micelle species. In combination with the microcentrifuge data, this allows for determination of free drug and colloidal content at the timepoint where both are tested.

The G-IB dissolution test is performed the same way, but with a 30 minute gastric exposure prior to transferring into the intestinal media. The intestinal media for the G-IB dissolution test is doubly concentrated in terms of buffering and bile salts, so that after the gastric transfer step, the final pH is 6.5 and SIF concentration is 0.5 wt %.

The microcentrifuge dissolution test separates the precipitate (undissolved drug) and total solubilized drug. Total solubilized drug consists of three solubilized species: freely solubilized drug, drug associated with bile salt micelles, and drug associated with polymer (drug-polymer colloids). These three species have different activities in vivo and differentiate formulations. The freely solubilized drug is the smallest species and is the most readily available for absorption. Freely solubilized drug can cross the unstirred mucous boundary layer and be absorbed into the blood through the epithelium. The drug associated with micelles diffuse across the unstirred mucous boundary layer and resupply free drug as it is absorbed. Finally, the drug-polymer colloids also resupply free drug as it is absorbed and can have a contribution to effective diffusion rate. These colloids can be important depending on the rate-limiting step for absorption.

In both dissolution tests, the solid dispersion formulations outperformed dissolution of Compound I alone with higher supersaturated solubility and better sustainability.

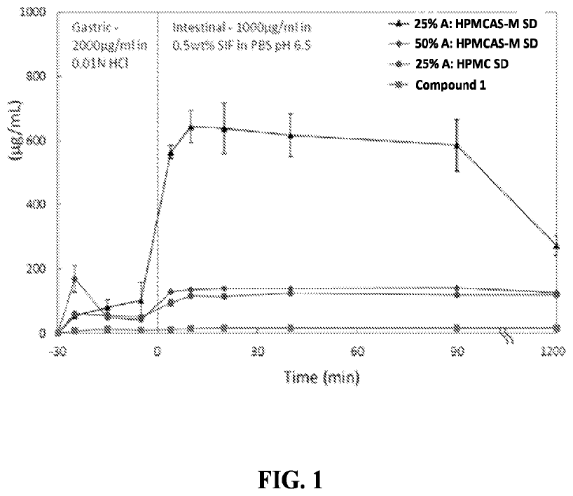

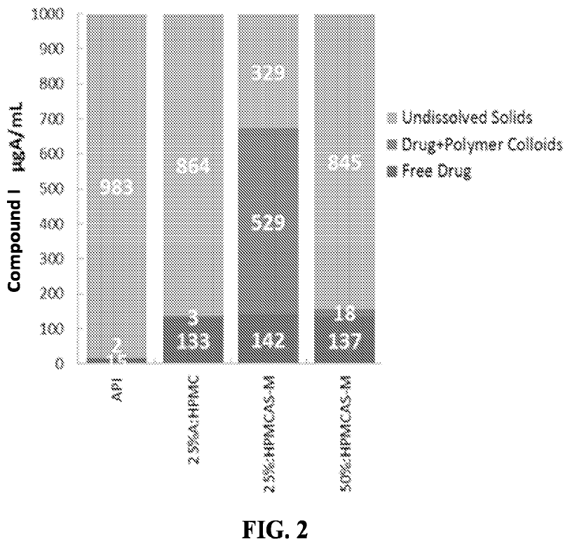

FIGS. 1, 2 show the results from the dissolution study. FIG. 1 illustrates dissolution data for the compositions, namely a solid dispersion (SD) having a weight ratio of 1:3 Compound I free base hydrate to HPMCAS (labeled as 25% HPMCAS); a solid dispersion (SD) having a weight ratio of 1:1 Compound I free base hydrate to HPMCAS (labeled as 50% HPMCAS); a solid dispersion (SD) having a weight ratio of 1:3 Compound I free base hydrate to HPMC (hydroxypropylmethyl cellulose, labeled as 25% HPMC), compared to Compound I free base hydrate alone. The error bars refer to standard deviation. As shown in FIG. 1, the solid dispersion of Compound I comprising Compound I and HPMCAS in a weight ratio of 1:3 (25% HPMCAS) had a superior dissolution profile compared to the other compositions under the test conditions.

FIG. 2 illustrates speciation data from the dissolution test. The solubility for amorphous Compound I was approximately 15 μg/mL. The free drug concentration of crystalline Compound I was similar to the solubility of amorphous Compound I. By contrast, the free drug concentration for Compound I in solid dispersions ranged from 133-142 μg/mL. For this test, all samples were analyzed for potency to ensure that all formulations were tested at similar concentrations. All of the solid dispersion formulations provided an enhancement in free drug concentration when compared with crystalline Compound I.

Surprisingly, it was also observed that the 25% Compound I: HPMCAS solid dispersion had a much higher concentration of drug in the form of drug-polymer colloids. Especially, a disproportionately high concentration (529 μg/mL) of drug-polymer colloid from the 25% Compound I:HPMCAS, compared to the drug-polymer colloid concentration (18 μg/mL) of the 50% Compound I:HPMCAS, was observed. The colloidal species increased the drug concentration to 671 μg/mL. approximately 500 μg/mL above the free drug value. As discussed above, the drug-polymer colloids is known to resupply free drug as it is absorbed, and can contribute to effective diffusion rate, and colloids can be important depending on the rate-limiting step for absorption. Accordingly, the surprisingly high concentration of drug in the form of drug-polymer colloids, in addition to drug in the free drug form would contribute to bioavailability of Compound I.

### Example 3-2: Dissolution Study

A solid dispersion comprising Compound I: HPMCAS in a ratio of 1:2 (“33.3% HPMCAS”) was prepared from 2.7 grams of the free base hydrate crystalline form B of Compound I and 5.3 grams of HPMCAS (Shin Etsu, L grade, F particle size (≤10 microns particle size)), using a similar procedure of Example 1. Briefly, 2 mg test compound/mL in 0.9 mL gastric buffer was transferred to a final dose of 1 mg/mL in 1.8 mL of 0.5% SIF in PBS at pH 6.5 at room temperature to provide 1.8 mL total volume test solution. The samples were centrifuged at 15,800 g for 1 minute. At specified timepoint, 50 μL aliquot samples were diluted 6× with 50/50 acetonitrile/water and assayed by HPLC.

The solid dispersion was evaluated together with the 25% Compound I: HPMCAS solid dispersion (“25% HPMCAS”) and 95.3% Compound I (freebase hydrate, “Crystalline API”), using the G-IB dissolution test described in Example 3-1. Results are shown in Table 2.

TABLE 2

| | C_{maxGB} | C_{max90 IB} | AUC_{4-90 IB} | C_{90 IB} | Ultra₉₀ | C₁₂₀₀ | Ultra₁₂₀₀ |
|---|---|---|---|---|---|---|---|
| Sample | (μg/mL) | (μg/mL) | (min*μg/mL) | (μg/mL) | (μg/mL) | (μg/mL) | (μg/mL) |
| 25% | 154 | 703 | 58,810 | 668 | 166 | 302 | 148 |
| HPMCAS | | | | | | | |
| 33.3% | 170 | 441 | 19,960 | 185 | 159 | 148 | 151 |
| HPMCAS | | | | | | | |
| Crystalline | 20 | 21 | 1,610 | 21 | 21 | 21 | 19 |
| API | | | | | | | |

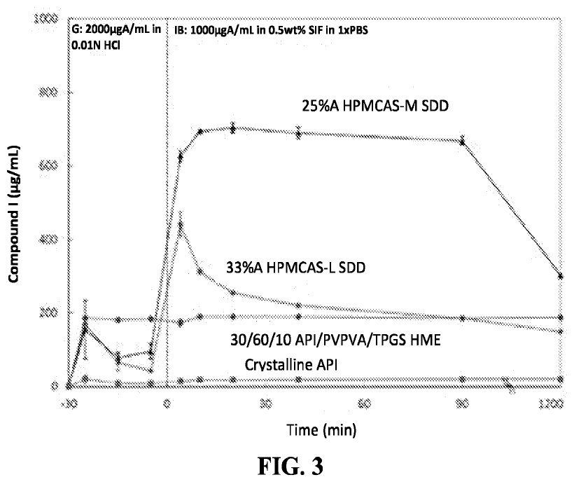

FIG. 3 shows the results from the dissolution study. Again, the solid dispersion of Compound I comprising Compound I and HPMCAS in a weight ratio of 1:3 (25% HPMCAS) had a superior dissolution profile compared to the other compositions under the test conditions.

### Example 4: Dog Pharmacokinetics (PK) Study

Compound I was administered orally in various amorphous spray dried dispersion preparations at a dose of 10 mg/kg to male beagle dogs (n=3). Compound I was administered via oral gavage (PO) at a dose volume of 0.4 mL/kg, in a 0.5% Methocel “A” suspension vehicle, at a concentration of 25 mg/mL, to yield a dose level of 10 mg/kg. The “0.5% Methocel “A” suspension vehicle” was prepared by warming 30 mL of deionized water to approximately 90° C. while stirring. Following removal from heat, 0.5 g of Methocel “A” (Dow Methocel A4M Premium) was added to the water and stirred until well dispersed, followed by 70 mL of deionized water and further stirring in an ice bath. The vehicle was stored under refrigerated conditions for up to one week. Compound I suspensions were prepared by placing the solid dispersion in a mortar and adding increasing volumes of 0.5% Methocel “A” suspension vehicle, with pestle mixing in-between. Suspensions were transferred into flasks and stirred for one minute.

Following dosing, blood samples (approximately 1.3 mL) were drawn into tubes containing potassium EDTA, via jugular vein, at 0.25, 0.5, 1, 2, 4, 6, 8 and 24 h. Samples were centrifuged (3,000×g at +4° C., 10 min) and the resultant plasma was separated from the erythrocyte pellet and stored at −20° C. prior to bioanalysis.

Compound I was extracted using protein precipitation. Plasma samples were thawed following storage at −20° C. and 50 μL aliquots precipitated with 150 μL acetonitrile containing an appropriate internal standard (IS) such as e.g., an analog of Compound I, e.g., a compound described in WO 2017/068412. Calibration standards and quality controls were prepared in plasma and extracted in the same manner. All samples were centrifuged at 4700 rpm, +4° C. for 20 minutes. Supernatant was analyzed using reverse-phase liquid chromatography mass spectrometry (LC-MS/MS).

Non-compartmental pharmacokinetic analyses were performed using Phoenix 6.3.0.395® (Certara USA, Inc) software. Calculated parameters included clearance, time of maximum observed concentration (T max), maximum concentration (Cmax), terminal half-life, area under the curve (AUC) from the time of dosing to the last measurable concentration (AUC₀₋ₜ) and extrapolated to infinity (AUC_{0-∞}).

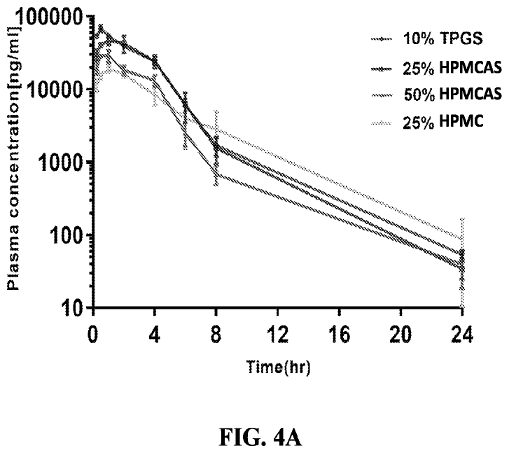

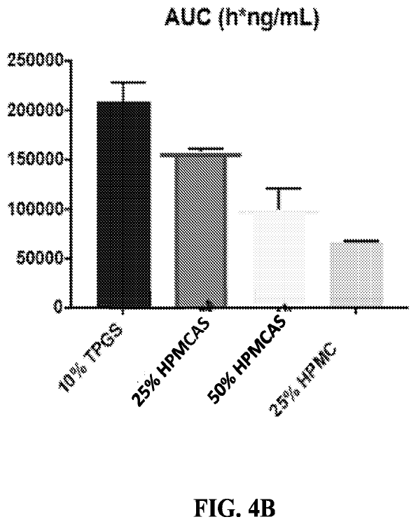

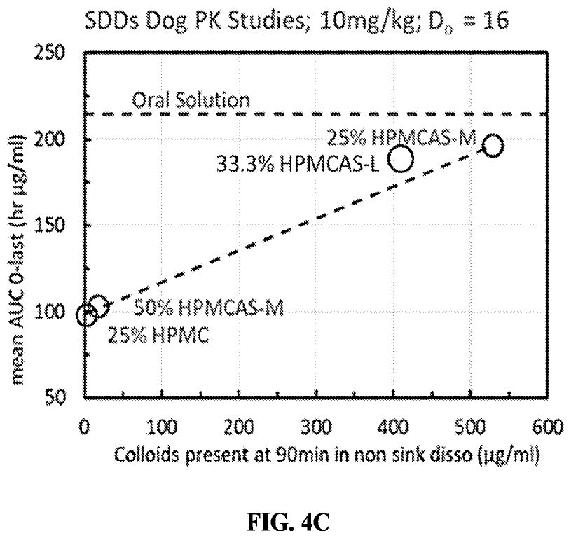

FIG. 4A shows plasma concentrations of Compound I and FIG. 4B shows the AUCs for the tested compositions in the dog PK study of this example. Tables 3, 4 below provides results from the dog PK study described in this example. Table 3 is a summary of mean plasma pharmacokinetic parameters of Compound I, following oral administration at 10 mg/kg as the spray dried dispersion preparation. Table 4 shows bioequivalence of Compound I exposure following oral administration at 10 mg/kg as the spray dried dispersion preparation, in various formulations. TPGS refers to D-α-tocopheryl polyethylene glycol 1000 succinate. The comparison formulation is a non-solid dispersion formulation.

TABLE 3

| | | | | | AUCₗₐₛₜ | AUC_{0-∞} |
|---|---|---|---|---|---|---|
| solid | | Tmax* | Cₘₐₓ | Half | (h · | (h · |
| dispersion | | (h) | (ng/mL) | Life (h) | ng/mL) | ng/mL) |
| 25% | Mean | 1.0 | 51090 | 2.2 | 196200 | 196300 |
| HPMCAS | SD | | 12560 | 0.20 | 67300 | 67300 |
| 50% | Mean | 1.0 | 31830 | 2.5 | 102800 | 103000 |
| HPMCAS | SD | | 5752 | 0.42 | 22790 | 22800 |
| 25% HPMC | Mean | 1.0 | 19310 | 2.6 | 98120 | 98540 |
| | SD | | 3989 | 0.73 | 56960 | 57640 |

*Tₘₐₓ expressed as median;
SD refers to Standard Deviation

TABLE 4

| | Comparison | | | |
|---|---|---|---|---|
| | formulation | | | |
| | 80% | | | |
| | Propylene | solid dispersion suspended | | |
| | glycol, 10% | in 0.5% Methocel “A” | | |
| | ethanol | 25% | 50% | 25% |
| Formulation | 10% TPGS | HPMCAS | HPMCAS | HPMC |
| Formulation conc | 100 | 25 | 25 | 25 |
| (mg/mL) | | | | |
| Dose vol (mL/kg) | 0.1 | 0.4 | 0.4 | 0.4 |
| Dose (mg/kg) | 10 | 10 | 10 | 10 |
| Concentration | 53792 | 29915 | 19821 | 11504 |
| (ng/ml) at 0.25 hr | | | | |
| Mean Cₘₐₓ (ng/mL) | 68028 | 51089 | 31829 | 19308 |
| Tmax(hr) | 0.5 | 0.25-2 | 0.5-1 | 1 |
| Mean AUCₗₐₛₜ | 214403 | 196181 | 102839 | 98119 |
| (hr*ng/mL) | | | | |
| Mean | 100 | 92 | 48 | 46 |
| Bioequivalence (%) | | | | |

As shown in Table 3, maximal exposure was obtained with the 25% HPMCAS-M batch, which showed comparable exposure to the 80% propylene glycol, 10% ethanol, 10% TPGS formulation. However, the 50% HPMCAS and 25% HPMC batches showed reduced oral exposure in comparison to both the 25% HPMCAS-M batch and the 80% propylene glycol, 10% ethanol, 10% TPGS formulations (Table 3).

This study showed that a solid dispersion of Compound I comprising Compound I and HPMCAS in a weight ratio of 1:3 had a desirable PK profile under the test conditions as demonstrated by the plasma concentrations and the AUCs and the Cmax. Unexpectedly the composition also showed less variability in exposure as shown by the smaller error bars for the 25% HPMCAS composition compared to the 50% HPMCAS composition I in FIG. 4B.

As shown in FIG. 4C, 50% HPMCAS-M and 25% HPMC test samples showed reduced oral exposure in comparison to both a 25% HPMCAS-M batch and a 33.33% HPMCAS-L batch. Test samples were dosed at 10 mg/Kg. Dₒ is calculated as follows:

$${D}_{o} = \frac{Dose / Volume\_GI}{Solubility} \\ {D}_{o} > 5 dinno rate should be considered during formulation$$

### Example 5: Primate Study

A total of 6 animals were used in a 3-way (2+2+2) crossover design where, on each of the three dosing occasions, 2 of the animals were administered Compound I in powder in bottle (PiB) suspension in vehicle for reconstitution (20 mg/kg), 2 of the animals were administered Compound I solid dispersion suspension (20 mg/kg) of Example 1, while another 2 animals were administered Compound I in a tablet form (20 mg/kg) of Example 2. The vehicle for reconstitution comprised Propylene Glycol USP 80.0% w/w; Ethanol USP 10.0% w/w; Vit E TPGS USP 10.0% w/w for a total of 100.0% w/w.

Dosing for this study was conducted in fasted state. On the second and third occasions of dosing (Day 8 and Day 15), the Compound I formulations for dosing of animals were reversed, respectively, so that at the end all six animals received Compound I in 3 different formulations (PiB in vehicle for reconstitution, solid dispersion suspension, or tablets) following completion of three occasions of oral administration. Solid dispersion suspension refers to a 5% suspension of the solid dispersion of Example 1 above in methocel.

The animals were observed closely during the first 12 hrs after each dosing and once daily on non-dosing days. Body weights were measured and recorded prior to dosing and blood samples for pharmacokinetic profiling were taken at the pre-defined time points for all animals. There were no clinical signs observed as a result of treatment with Compound I. Oral administration of Compound I in powder-in-bottle formulation, solid dispersion suspension, or tablet form at 20 mg/kg, three dosing occasions 7 days apart was well tolerated in monkeys. Before sample analysis, the partial qualification of the method to determine the concentration of Compound I in monkey plasma by LC-MS/MS was conducted successfully over a dynamic assay range of 1-2,000 ng/mL. All plasma samples from this study were analyzed by this method and plasma concentration of Compound I were expressed in terms of ng/mL.

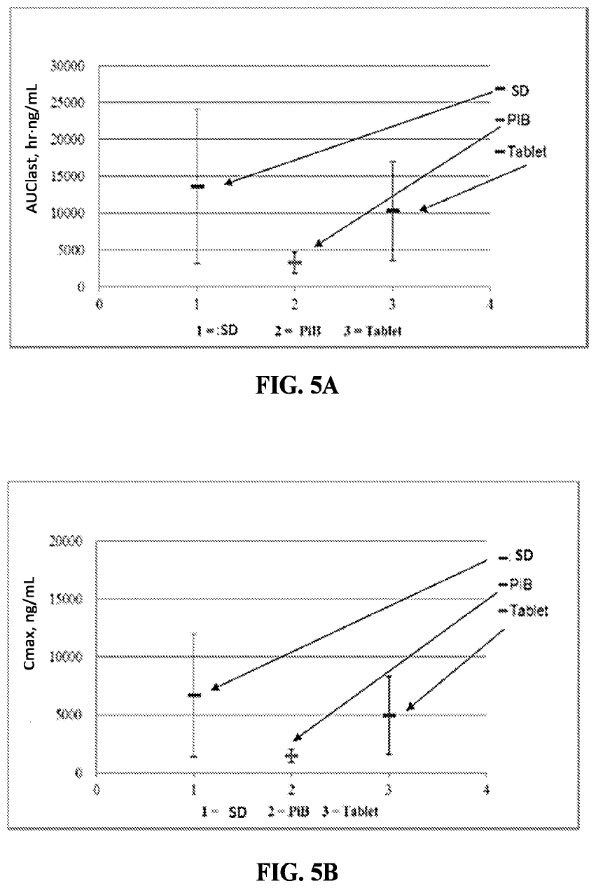

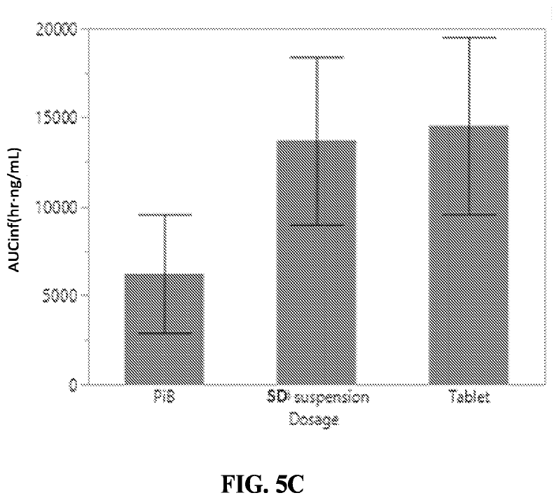

FIG. 5A shows plasma concentrations (AUCₗₐₛₜ) of Compound I, FIG. 5B shows plasma concentrations (Cₘₐₓ) of Compound I, and FIG. 5C shows the AUCs (inferred) for the tested compositions in the primate PK study of this example. The error bars refer to standard deviation. SD refers to solid dispersion. PiB refers to powder in a bottle. Tablet refers to the tablet composition of Example 2 above. AUCₗₐₛₜ refers to the area under the concentration-time curve from dosing (time 0) to the time of the last measured concentration (24 hours).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed:

1. A pharmaceutical composition comprising Compound I, having the formula: 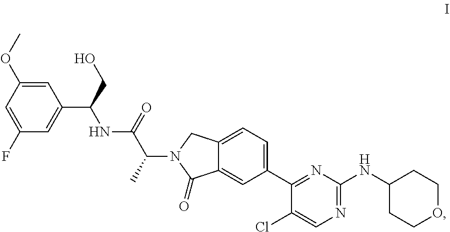 [SMILES: COc1cc(F)cc([C@@H](CO)NC(=O)[C@@H](C)N2Cc3ccc(-c4nc(NC5CCOCC5)ncc4Cl)cc3C2=O)c1]
or a pharmaceutically acceptable salt or solvate thereof, and a water soluble polymer.

2. The composition of claim 1, wherein the water soluble polymer is polyvinylpyrrolidone/vinyl acetate (PVPVA), hydroxypropylmethyl cellulose (HPMC), or hydroxypropylmethyl cellulose acetate succinate (HPMCAS).

3. The composition of claim 1, wherein the water soluble polymer is hydroxypropylmethyl cellulose acetate succinate (HPMCAS).

4. The composition of claim 1, comprising Compound I free base hydrate.

5. The composition of claim 1, comprising Compound I free base monohydrate.

6. A solid dispersion comprising Compound I, having the formula: 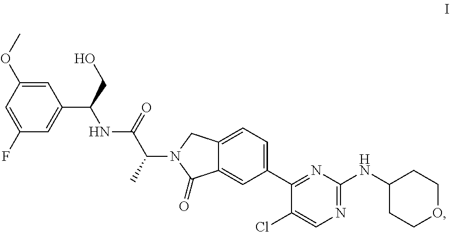 [SMILES: COc1cc(F)cc([C@@H](CO)NC(=O)[C@@H](C)N2Cc3ccc(-c4nc(NC5CCOCC5)ncc4Cl)cc3C2=O)c1]
or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS).

7. The solid dispersion of claim 6, wherein Compound I, or a pharmaceutically acceptable salt or solvate thereof, is present in the solid dispersion in an amount of about 20% to about 50% by weight of the solid dispersion.

8. The solid dispersion of claim 6, wherein Compound I, or a pharmaceutically acceptable salt or solvate thereof, is present in the solid dispersion in an amount of about 20% to about 40% by weight of the solid dispersion.

9. The solid dispersion of claim 6, wherein Compound I, or a pharmaceutically acceptable salt or solvate thereof, is present in the solid dispersion in an amount of about 20% to about 30% by weight of the solid dispersion.

10. The solid dispersion of claim 6, wherein Compound I, or a pharmaceutically acceptable salt or solvate thereof, is substantially amorphous.

11. The solid dispersion of claim 6, wherein at least 98% of Compound I, or a pharmaceutically acceptable salt or solvate thereof, is in amorphous form.

12. The solid dispersion of claim 6, wherein Compound I is a free base or a solvate thereof.

13. The solid dispersion of claim 6, wherein Compound I is a free base hydrate.

14. The solid dispersion of claim 6, wherein the ratio of an amount by weight of Compound I, or a pharmaceutically acceptable salt or solvate thereof, in the solid dispersion to an amount by weight of HPMCAS in the solid dispersion is from about 1:1 to about 1:5.

15. The solid dispersion of claim 6, wherein the ratio of an amount by weight of Compound I, or a pharmaceutically acceptable salt or solvate thereof, in the solid dispersion to an amount by weight of HPMCAS in the solid dispersion is from about 1:2 to about 1:4.

16. The solid dispersion of claim 6, wherein the ratio of an amount by weight of Compound I in the solid dispersion to an amount by weight of HPMCAS in the solid dispersion is about 1:3.

17. The solid dispersion of any one of claims 6-16, wherein HPMCAS is present in an amount greater than about 50% by weight of the solid dispersion.

18. The solid dispersion of any one of claims 6-16, wherein HPMCAS is present in an amount of about 60% to about 80% by weight of the solid dispersion.

19. A pharmaceutical composition comprising the solid dispersion of any one of claims 6-18.

20. The pharmaceutical composition of claim 19, wherein the composition is formulated as a tablet.

21. The pharmaceutical composition of any one of claims 19-20, wherein the composition is formulated as a tablet comprising an intra-granular layer and an extra-granular layer.

22. The pharmaceutical composition of any one of claims 19-22, wherein the solid dispersion comprises about 40%-70% by weight of the tablet.

23. The pharmaceutical composition of any one of claims 19-22, wherein the solid dispersion comprises about 60% by weight of the tablet.

24. The pharmaceutical composition of any one of claims 19-23, wherein the intra-granular layer of the tablet further comprises:
about 8-18% by weight of microcrystalline cellulose;
about 8-18% by weight of lactose monohydrate;
about 2-8% by weight of croscarmellose sodium;
about 0.5-2% by weight of non-fumed silica; and
about 0.1-0.5% by weight of magnesium stearate.

25. The pharmaceutical composition of any one of claims 19-24, wherein the extra-granular layer of the tablet comprises:
about 0-14% by weight of microcrystalline cellulose;
about 0-14% by weight of lactose monohydrate;
about 2-8% by weight of croscarmellose sodium;
about 0.5-2% by weight of non-fumed silica; and
about 0.1-0.5% by weight of magnesium stearate.

26. The pharmaceutical composition of any one of claims 19-25, wherein the table is coated with a film.

27. A tablet comprising a solid dispersion, wherein the solid dispersion comprises Compound I, having the formula: 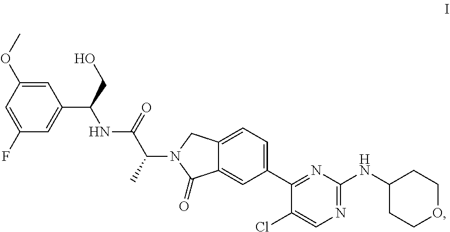 [SMILES: COc1cc(F)cc([C@@H](CO)NC(=O)[C@@H](C)N2Cc3ccc(-c4nc(NC5CCOCC5)ncc4Cl)cc3C2=O)c1]
or a pharmaceutically acceptable salt or solvate thereof,
wherein Compound I, or a pharmaceutically acceptable salt or solvate thereof, is molecularly dispersed within a polymer matrix comprising hydroxypropylmethyl cellulose acetate succinate (HPMCAS); and the ratio of an amount by weight of Compound I, or a pharmaceutically acceptable salt or solvate thereof, in the solid dispersion to an amount by weight of HPMCAS in the solid dispersion is from about 1:1 to about 1:5.

28. The tablet of claim 27, wherein the ratio of an amount by weight of Compound I, or a pharmaceutically acceptable salt or solvate thereof, in the solid dispersion to an amount by weight of HPMCAS in the solid dispersion is from about 1:2 to about 1:4.

29. The tablet of claim 27, wherein the ratio of an amount by weight of Compound I in the solid dispersion to an amount by weight of HPMCAS in the solid dispersion is about 1:3.

30. The tablet of claim 27, wherein Compound I, or a pharmaceutically acceptable salt or solvate thereof, is substantially amorphous.

31. The tablet of claim 27, wherein at least 98% of Compound I, or a pharmaceutically acceptable salt or solvate thereof, is in amorphous form.

32. The tablet of claim 27, wherein Compound I is a free base or a solvate thereof.

33. The tablet of claim 27, wherein Compound I is a free base hydrate.

34. The tablet of claim 27, wherein Compound I is a free base monohydrate.

35. A tablet composition of Compound I having the formula: 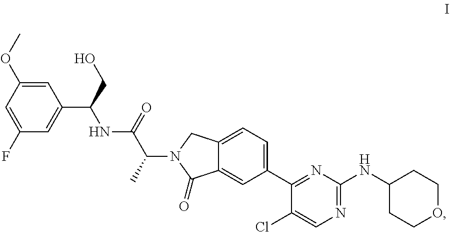 [SMILES: COc1cc(F)cc([C@@H](CO)NC(=O)[C@@H](C)N2Cc3ccc(-c4nc(NC5CCOCC5)ncc4Cl)cc3C2=O)c1]
or a pharmaceutically acceptable salt or solvate thereof,
comprising:
an intra-granular portion comprising:
about 60% by weight of a solid dispersion of Compound I, or a pharmaceutically acceptable salt or solvate thereof, and HPMCAS, wherein Compound I, or a pharmaceutically acceptable salt or solvate thereof, is substantially amorphous and molecularly dispersed within a polymer matrix comprising hydroxypropylmethyl cellulose acetate succinate (HPMCAS), and the ratio of an amount by weight of Compound I, or a pharmaceutically acceptable salt or solvate thereof, in the solid dispersion to an amount by weight of HPMCAS in the solid dispersion is about 1:3;
about 14% by weight of microcrystalline cellulose;
about 13.5% by weight of lactose monohydrate;
about 6% by weight of croscarmellose sodium;
about 1% by weight of non-fumed silica; and
about 0.25% by weight of magnesium stearate; and
an extra-granular portion comprising:
about 4% by weight of croscarmellose sodium;
about 1% by weight of non-fumed silica; and
about 0.25% by weight of magnesium stearate.

36. The tablet composition of claim 35, wherein the HPMCAS has an acetyl content in a range of about 7 to about 11 percent by weight, and succinoyl content in a range of about 10 to about 14 percent by weight of the HPMCAS.

37. Use of a composition of any one of claims 1-36 for treating cancer.

38. A method of treating cancer comprising administering a composition of any one of claims 1-36 to an individual in need thereof.

39. A process for preparing a solid dispersion of claim 6 comprising spray drying a solution of Compound I, or a pharmaceutically acceptable salt or solvate thereof, and HPMCAS in a solvent, wherein the solution has a solids loading of up to about 25% by weight.

40. The process of claim 39, wherein the solvent is acetone or methanol.

41. The process of claim 39, wherein the solid dispersion is prepared by spray drying a solution of Compound I, or a pharmaceutically acceptable salt or solvate thereof, and HPMCAS in acetone, wherein the acetone solution has a solids loading of up to about 10% by weight.

42. The process of claim 39, wherein Compound I is a free base hydrate.

43. The process of claim 39, wherein Compound I is a free base monohydrate.
